(19) United States
(12) Patent Application Publication
(10) Pub. No.: US 2019/0134234 A1
(43) Pub. Date: May 9, 2019

(54) COMPOSITION FOR STABILIZING RADIOCHEMICAL PURITY OF [18F] FLUORO-DOPA AND METHOD FOR PREPARING SAME

(71) Applicant: CI-CO HEALTHCARE CO., LTD., Seoul (KR)

(72) Inventors: Sang Ju LEE, Seoul (KR); Seung Jun OH, Seoul (KR); Jin Sook RYU, Seoul (KR); Jae Seung KIM, Seoul (KR); Dae Hyuk MOON, Seoul (KR); Jong Jin LEE, Seoul (KR)

(73) Assignee: CI-CO HEALTHCARE Co., Ltd., Seoul (KR)

(21) Appl. No.: 15/746,673

(22) PCT Filed: Jul. 22, 2016
(86) PCT No.: PCT/KR2016/008049

§ 371 Date: Jan. 22, 2018

(30) Foreign Application Priority Data
Jul. 22, 2015 (KR) 10-2015-0103996
Jul. 21, 2016 (KR) 10-2016-0092604

(51) Int. Cl. A61K 51/04

(52) U.S. Cl. CPC A61K 51/0402

(57) ABSTRACT

The present disclosure relates to a composition for stabilizing the radiochemical purity of [¹⁸F]fluoro-dopa by inhibiting the formation of impurities during a predetermined time period, and a method for preparing the same. The composition according to the present disclosure includes: [¹⁸F]fluoro-dopa; ethanol in a concentration of 5% to 30%(v/v) relative to the total composition; and a buffer having a pKa value of 6 to 8.1 at 25° C., in which the composition stabilizes the radiochemical purity of the [¹⁸F]fluoro-dopa by inhibiting the formation of impurities during a predetermined time period.

### TECHNICAL FIELD

The present disclosure relates to a composition for stabilizing the radiochemical purity of [¹⁸F]fluoro-dopa and a method for preparing the same, and more particularly to a composition for stabilizing the radiochemical purity of [¹⁸F]fluoro-dopa by inhibiting the formation of impurities during a predetermined time period, and a method for preparing the same.

### BACKGROUND ART

Recently, for early diagnosis of brain diseases such as Parkinson's disease, depression, schizophrenia, and Alzheimer's disease; heart disease due to stress and dietary changes; and diseases such as various cancers caused by exposure to various harmful substances of the human body, various image diagnosis methods have been used. Positron emission tomography (PET) is widely used as a method that is directly applicable to clinical practices. The positron emission tomography is a method of imaging the distribution and biochemical change process of a radiopharmaceutical in a living body by intravenously injecting an organic compound labeled with a radioisotope that emits positron in vivo. Therefore, positron emission tomography can quantitatively measure the biochemical changes of a living body at the lesion site, so that the disease progression can be measured and the degree of treatment can be predicted (A. Agool, R. H. Slart, K. K. Thorp, A. W. Glaudemans, D. C. Cobben, L. B. Been, F. R. Burlage, P. H. Elsinga, R. A. Dierckx, E. Vellenga, J. L. Holter, Nucl. Med. Commun. 2011, 32, 14.; N. Aide, K. Kinross, C. Cullinane, P. Roselt, K. Waldeck. O, Neels, D. Dorow, G. McArthur, R. J. Hicks, J. Nucl. Med. 2011, 51, 1559.; A. Debucquoy, E. Devos, P. Vermaelen, W. Landuyt, S. De Weer, F. Van Den Heuvel, K. Haustermans, Int. J. Radiat. Biol. 2009, 85, 763.).

The radioisotope used in the positron emission tomography is fluoride ([¹⁸F]F), carbon ([¹⁵C]C), nitrogen ([¹³N]N), oxygen ([¹⁵O]O) and gallium (⁶⁸Ga)Ga). Among them, [¹⁸F]fluoride has a size similar to hydrogen, forms a stable bond with the carbon of an organic compound, and can be easily produced. It is reported that it has a suitable half-life (110 minutes), and thus is very suitable in performing positron emission tomography (Lasne, M. C.; Perrio, C.; Rouden, J.; Bane, L.; Roeda, D.; Dolle, F.; Crouzel, C. Contrast Agents II, Topics in Current Chemistry, Springer-Verlag, Berlin, 2002, 222, 201-258.; Bolton, R. J. Labelled Compd. Radiopharm. 2002, 45, 485-528). [¹⁸F]fluoride is generally prepared by irradiating a proton to [¹⁸O]H2O with a cyclotron as a circular accelerator (M. R. Kilbourn, J. T. Hood, M. J. Welch, Int. J. Appl. Radiat. Isot. 1984, 35, 599.; G. K. Mulholland, R. D. Hichwa, M. R. Kilbourn, J. Moskwa, J. Label. Compd. Radiopharm. 1989, 26, 140.).

PET or PET/CT imaging with the use of [¹⁸F]fluoro-dopa(¹⁸F-FLUORODOPA, [¹⁸F]FLUORODOPA, ¹⁸F-6-L-FLUORODOPA, [¹⁸F]-FLUORO-L-DOPA, L-6-[¹⁸F]FLUORO-3,4-DIHYDROXYPHENYLALANINE, FDOPA), which is a radiopharmaceutical for positron emission tomography, is used to measure the disappearance of dopamine nerve distal end in the brain striatum. This test method can be used to diagnose Parkinson's disease and differentiate between essential tremor and Parkinson's syndrome (Fischman, A. Radiol. Clin. N. Am. 2005, 43, 93-106; Vingerhoets, F.J.; Schulzer, M.; Ruth, T. J.; Holden, J. E.; Snow, B. J. J. Nucl. Med. 1996, 37, 421-426.; Sawle, G. W.; Wroe, S. J.; Lees, A. J.; Brooks, D. J.; Frackowiak, R. S.; Ann. Neurol. 1992, 32, 609-617). In addition, it is possible to measure the pathophysiological function of tissues or organs with increased intracellular transport and decarboxylation of dihydroxyphenylalanine, which is an amino acid, and it is used for diagnosis and localization of insulin species accompanied by hyperinsulinism in infants and children (Tessonnier, L.; Sebag, F.; Ghander, C.; De Micco, C.; Reynaud, R.; Palazzo, F. F.; Conte-Devolx, B.; Henny, J. F.; Mundler, O.; Taieb, D. J. Clin. Endocrinol Metab. 2010, 95, 303-307; Ribeiro, M. J.; De Lonlay, P.; Delzescaux, T.; Boddaert, N.; Jaubert, F.; Bourgeois, S.; Dolle, F; Nihoul- Fekete, C.; Syrota, A.; Brunelle, F. J. Nucl. Med. 2005, 46, 560-566).

FDOPA is fluorinated L-DOPA, and the biological classification of FDOPA is similar to that of L-DOPA (http://interactive.snm.org/docs/PET_PROS/FDOPA.pdf).

On the other hand, it is known that L-DOPA is unstable in aqueous alkaline solution and oxidized by oxygen https://www.sigmaaldrich.com/content/dam/sigmaaldrich/docs/Sigma/Product_Information_She et/d9628pis.pdf), and the storage temperature is known as 2 to 8° C. (http://www.sigmaaldrich.com/catalog/product/aldrich/333786lang=ko®ion=KR). [¹⁸F]fluoro-dopa, which has been reported to date by these chemical properties of L-DOPA, is also difficult to store and use. [¹⁸F]fluoro-dopa is stable in an acidic state and is unstable in a neutralized state. Thus, it is known that it is distributed in an acidic state and neutralized immediately before use. The pH in the United States Pharmacopeia (USP) and European Pharmacopoeia (EP) is set at 4.0 to 5.5 (USP31/NF26, vol 2. 2008. 2193-2194.; EP 6.0, 2008. 990-992,; Kao, C. H.; Hsu, W. L.; Xie, H. L.; Lin, M. C.; Lan, W. C.; Chao, H. Y.; Ann Nucl Med. 2011, 25, 309-316). However, in the case of IASOdopa manufactured and distributed in the Europe, the pH is in a strongly acidic condition of 2.3 to 3.0 when the drug is taken out. Also, referring to the IASOdopa SPC (summary of product characteristics, http://agence-prd.ansm.sante.fr/html/par_eu/20080604_fr328_iasodopa_spc.pdf), it is stated that it should be stored at 2to 8° C. after pH neutralization, and should be used within 2 hours after neutralization. It is described that [¹⁸F]fluoro-dopa is oxidized at a pH of 4.5 or higher. Therefore, due to the half-life of the radioisotope, (18F, 110 minutes), radiopharmaceuticals should be manufactured and used whenever necessary. The radioactive content of the radiopharmaceutical may be different whenever it is manufactured due to the difference in the amount and the yield of the starting radioactivity. Due to the half-life characteristics after manufacture, the volume of the radiopharmaceutical administered to a patient increases over time. In addition, [¹⁸F]fluoro-dopa is stored at an acidic pH, so it must be neutralized for administration to the human body. If a patient is injected with a great quantity in a strongly acidic condition without neutralization, there is a risk of causing metabolic acidosis.

Therefore, when [¹⁸F]fluoro-dopa is used for PET diagnosis, there is a considerable inconvenience in use because there is a time constraint depending on half-life of a radioisotope, there are restrictions that neutralization is necessarily required for the administration of [¹⁸F]fluoro-dopa stored in an acidic condition to the human body, there are restrictions on the use time required to be administered to the human body within 2 hours after neutralization, and there are restrictions that it is necessary to keep it refrigerated.

Prior art documents related to the present disclosure include Korean Patent Application No. 10-2007-7020237 (Title of the invention: New Pharmaceutical Compositions Useful in the Treatment of Parkinson's Disease, filing date: September 4, 2007).

### DISCLOSURE

### Technical Problem

In order to solve this problem, there are provided a composition for stabilizing the radiochemical purity of [¹⁸F]fluoro-dopa which has radiochemical purity that can be used as a radiopharmaceutical also for 2 to 6 hours after the synthesis of [¹⁸F]fluoro-dopa, and a method for preparing the same.

In addition, there are provided a composition for stabilizing the radiochemical purity of [¹⁸F]fluoro-dopa which has radiochemical purity that can be used as a radiopharmaceutical without oxidation even at a neutral pH, preferably pH 6 to 8, not at an acidic pH, even for 2 to 6 hours after the synthesis of [¹⁸F]fluoro-dopa, and a method for preparing the same.

In addition, there are provided a composition for stabilizing the radiochemical purity of [¹⁸F]fluoro-dopa having radiochemical purity which can be used as a radiopharmaceutical even at a room temperature without refrigeration, and a method for preparing the same.

### Technical Solution

In order to achieve the above object, according to the present disclosure, there is provided a composition for stabilizing the radiochemical purity of [¹⁸F]fluoro-dopa by inhibiting the formation of impurities during a predetermined time period, comprising: [18F]fluoro-dopa; ethanol in a concentration of 5% to 30%(v/v) relative to the total composition; and a buffer having a pKa value of 6 to 8.1 at 25° C.

The composition exhibits a radiochemical purity of at least 90% by inhibiting the formation of impurities for 2 to 6 hours after synthesis of the [¹⁸F]fluoro-dopa at a pH of 6 to 8.

The composition inhibits the formation of impurities for 2 to 6 hours after synthesis of the [¹⁸F]fluoro-dopa at room temperature to exhibit a radiochemical purity of at least 90%.

The buffer having a pKa value of 6.1 to 8.1 at 25° C. is at least one of those selected from the group consisting of PBS(PHOSPHATE BUFFERED SALINE), CITRATE BUFFER, MES(2-(NMORPHOLINO)ETHANESULFONIC ACID), BIS-TRIS (2,2-BIS(HYDROXYMETHYL)-2,2′,2″-NITRILOTRIETHANOL) buffer, MOPSO (3-MORPHOLINO-2-HYDROXYPROPANESULFONIC ACID) buffer, HEPES(4-(20HYDROXETHYL)-1-PIPERAZINEETHANSFULFONIC ACID) buffer and TRIS (TRIS (HYDROXYMETHYL)AMINOMETHANE) buffer.

The dopa includes levodopa (L-dopa), carbadopa, dopamine or derivatives thereof. In a composition for stabilizing the radiochemical purity of the [¹⁸F]fluoro-dopa according to the present disclosure, the [¹⁸F]fluoro-dopa is included at a radioactive concentration of 37 to 37,000 MBq/ml and ethanol and the buffer are included such that the total volume of the buffer is 100% (V/V) of the total composition. For example, the ethanol is included at a concentration of 5% to 30% (V/V) with respect to the total composition and the buffer is included at 70% to 95% with respect to the total composition, so that the sum of the buffer concentration and the ethanol concentration can be 100% (V/V).

Further, in order to achieve the above object, according to the present disclosure, there is provided a method for preparing a composition for stabilizing the radiochemical purity of [¹⁸F]fluoro-dopa by inhibiting the formation of impurities during a predetermined time period, comprising: mixing [18F]fluoro-dopa; ethanol in a concentration of 5% to 30%(v/v) relative to the total composition; and a buffer having a pKa value of 6 to 8.1 at 25° C.

The composition exhibits a radiochemical purity of at least 90% by inhibiting the formation of impurities for 2 to 6 hours after synthesis of the [¹⁸F]fluoro-dopa at a pH of 6 to 8.

The composition inhibits the formation of impurities for 2 to 6 hours after synthesis of the [¹⁸F]fluoro-dopa at room temperature to exhibit a radiochemical purity of at least 90%.

The buffer having a pKa value of 6.1 to 8.1 at 25° C. is at least one of those selected from the group consisting of PBS(PHOSPHATE BUFFERED SALINE), CITRATE BUFFER, MES(2-(NMORPHOLINO)ETHANESULFONIC ACID), BIS-TRIS (2,2-BIS(HYDROXYMETHYL)-2,2′,2″-NITRILOTRIETHANOL) buffer, MOPSO (3-MORPHOLINO-2-HYDROXYPROPANESULFONIC ACID) buffer, HEPES(4-(20HYDROXETHYL)-1-PIPERAZINEETHANSFULFONIC ACID) buffer and TRIS(TRIS(HYDROXYMETHYL)AMINOMETHANE) buffer.

The dopa includes levodopa (L-dopa), carbadopa, dopamine or derivatives thereof.

### Advantageous Effects

According to the present disclosure, there are provided a composition for stabilizing the radiochemical purity of [¹⁸F]fluoro-dopa which has radiochemical purity that can be used as a radiopharmaceutical also for 2 to 6 hours after the synthesis of [¹⁸F]fluoro-dopa, and a method for preparing the same.

In addition, there are provided a composition for stabilizing the radiochemical purity of [¹⁸F]fluoro-dopa which has radiochemical purity that can be used as a radiopharmaceutical without oxidation even at a neutral pH, preferably pH 6 to 8, not at an acidic pH, even for 2 to 6 hours after the synthesis of [¹⁸F]fluoro-dopa, and a method for preparing the same.

In addition, there are provided a composition for stabilizing the radiochemical purity of [¹⁸F]fluoro-dopa having radiochemical purity which can be used as a radiopharmaceutical even at a room temperature without refrigeration, and a method for preparing the same.

### DESCRIPTION OF DRAWINGS

FIG. 1 is an HPLC chromatogram of an ethanol-free[¹⁸F]fluoro-dopa agent illustrating the formation of impurities at 2 hours after neutralization;

FIG. 2 is an HPLC chromatogram of an ethanol-free[¹⁸F]fluoro-dopa agent illustrating the formation of impurities at 6 hours after neutralization;

FIG. 3 is a graph illustrating the radiochemical purity of an [¹⁸F]fluoro-dopa agent added with and without ethanol after neutralization at a pH of 6;

FIG. 4 is a graph illustrating the radiochemical purity of the [¹⁸F]fluoro-dopa agent added with and without ethanol after neutralization at a pH of 7;

FIG. 5 is a graph illustrating the radiochemical purity of the [¹⁸F]fluoro-dopa agent added with and without ethanol after neutralization at a pH of 8;

FIG. 6 is a TLC chromatogram of an [¹⁸F]fluoro-dopa agent added with 10% ethanol illustrating the formation of impurities at 1 hour after neutralization;

FIG. 7 is a graph illustrating the radiochemical purity of an [¹⁸F]fluoro-dopa agent added with and without ethanol, depending on the storage temperature after neutralization;

FIG. 8 is a photograph of a PET image of an [¹⁸F]fluoro-dopa agent added with ethanol and without buffer as time passes after neutralization;

FIG. 9 is a graph illustrating the radiochemical purity of an [¹⁸F]fluoro-dopa agent added with ethanol and buffer as time passes after neutralization.

### BEST MODE

Hereinafter, the present disclosure will be described in detail so as to be easily carried out by those skilled in the art. The present disclosure may be embodied in many different forms and is not limited to the examples described herein. In order to clearly illustrate the present disclosure, parts that are not related to the description are omitted, and the same or similar components are denoted by the same reference numerals throughout the specification.

The present disclosure provides a composition for stabilizing the radiochemical purity of [¹⁸F]fluoro-dopa by inhibiting the formation of impurities during a predetermined time period, the composition comprising: [18F]fluoro-dopa; ethanol in a concentration of 5% to 30%(v/v) relative to the total composition; and a buffer having a pKa value of 6 to 8.1 at 25° C.

[¹⁸F]fluoro-dopa includes levodopa (L-dopa), carbidopa, dopamine or derivatives thereof. It is preferably prepared by labeling levodopa (L-DOPA) with [¹⁸F]fluoride through a nucleophilic substitution reaction, and the labeling method follows known procedures (Source: Appl. Radiat. Isot. 67 2009 1650-1653).

The [¹⁸F]fluoride source may be a fluorine salt, which is a compound including fluorine-18, which may be a known fluorine salt. Or, the labeling of The [¹⁸F]fluoride can achieve radioactive labeling with fluorine by electrophilic fluorination using ¹⁸ [F]F fluorine gas. Preferably, it can be achieved by nucleophilic substitution of the appropriate leaving group by [¹⁸F]-fluoride ion.

Thus, [¹⁸F]fluoro-dopa retains the chemical properties of levodopa (L-DOPA). Levodopa is unstable in aqueous alkaline solution, oxidizes well by oxygen, and should be stored at 2 to 8° C. (see: https://www.sigmaaldrich.com/content/dam/sigmaaldrich/docs/Sigma/Product_Information_Shee t/d9628pis.pdf; http://www.sigmaaldrich.com/catalog/product/aldrich/3337861ang=ko&region=KR). Therefore, the [¹⁸F]fluoro-dopa reported to date by these chemical properties of levodopa is also difficult to store and use. Since the [¹⁸F]fluoro-dopa is unstable in the neutral state, it is stored and distributed in an acidic state and a refrigerated state. It has been known that it should be in a neutralized state for administration in the human body, and thus should be neutralized immediately before use and used within 2 hours after neutralization. This is also stated in the [¹⁸F]fluoro-dopa product IASOdopa SPC (summary of product characteristics, http://agenceprd.ansm.sante.fr/html/par_eu/20080604_fr328_iasodopa_spc.pdf), manufactured and sold in the Europe It is stated herein that after pH neutralization, it must be stored at 2 to 8° C. and used within 2 hours after neutralization and it is stated that [¹⁸F]fluoro-dopa is oxidized at a pH of 4.5 or higher.

On the other hand, [¹⁸F]fluoro-dopa is decomposed by non-enzymatic oxidation at the elapse of 2 hours after the synthesis, and there is also a thesis on a method for preventing this (see: J NUCL MED 30: 1249-1256, 1989). The chemical properties of this [¹⁸F]fluoro-dopa are different from [¹⁸F]FDG or [¹⁸F]FLT, which are commonly used as PET imaging agents. In case of [¹⁸F]FDG or [¹⁸F]FLT, the chemical properties are not affected by pH.

The inventors of the present disclosure found that the reasons for being greatly affected by the storage pH, the use time, and the storage temperature after the synthesis of [¹⁸F]fluoro-dopa were because radiochemical impurities are generated which affect the radiochemical purity after the synthesis of [¹⁸F]fluoro-dopa. Accordingly, as a result that the inventors of the present disclosure have conducted studies on a [¹⁸F]fluoro-dopa preparation capable of neutral pH, prolonged use time after neutralization, and storage at room temperature after neutralization, they confirmed that ethanol in a concentration of 5% to 30% (V/V) relative to the total composition; and a buffer having a pKa value of 6 to 8.1 at 25° C. could inhibit the formation of radiochemical impurities, thereby completing the present disclosure.

Ethanol can be purchased and used from a manufacturer with ethanol having a purity of 100% and can be used in a concentration of 5% to 30% (V/V) relative to the total composition. The formation of the first and second radiochemical impurities, which are affected by the hydrogen ion concentration and the storage time, detected by HPLC after neutralization from the [¹⁸F]fluoro-dopa synthesized by ethanol addition is inhibited so that the radiochemical purity of [¹⁸F]fluoro-dopa can be maintained at least 90% or more.

A buffer having a pKa value of 6 to 8.1 at 25° C. can also be purchased and used from a manufacturer. The buffer having a pKa value of 6.1 to 8.1 at 25° C. is at least any one of those selected from the group consisting of PBS(PHOSPHATE BUFFERED SALINE), CITRATE BUFFER, MES(2-(NMORPHOLINO)ETHANESULFONIC ACID), BIS -TRIS (2,2-BIS(HYDROXYMETHYL)-2,2′,2″-NITRILOTRIETHANOL) buffer, MOPSO (3-MORPHOLINO-2-HYDROXYPROPANESULFONIC ACID) buffer, HEPES(4-(20HYDROXETHYL)-1-PIPERAZINEETHANSFULFONIC ACID) buffer and TRIS(TRIS(HYDROXYMETHYL)AMINOMETHANE, or TRIZMA®) buffer.

The addition of the buffer of the above condition inhibits the formation of the third radiochemical impurity, which is affected by the storage temperature, detected by TLC after neutralization from the [¹⁸F]fluoro-dopa so that the radiochemical purity of [¹⁸F]fluoro-dopa can be maintained at least 90% or more.

Accordingly, the composition according to the present disclosure inhibits the formation of impurities for 2 to 6 hours after the synthesis of the [¹⁸F]fluoro-dopa at a pH of 6 to 8 to exhibit a radiochemical purity of at least 90%.

In addition, the composition according to the present disclosure inhibits the formation of impurities for 2 to 6 hours after the synthesis of the [¹⁸F]fluoro-dopa at room temperature to exhibit a radiochemical purity of at least 90%.

In addition, the composition according to the present disclosure inhibits the formation of impurities for 2 to 6 hours after the synthesis of the [¹⁸F]fluoro-dopa at a pH of 6 to 8 and at room temperature to exhibit a radiochemical purity of at least 90%.

On the other hand, the [¹⁸F]fluoro-dopa prepared by the above known synthesis method is in an acidic state of pH 2 to 4 and needs to be adjusted to a pH suitable for human or mammalian administration, for example, a pH of 6 to 8. Sodium bicarbonate, sodium carbonate or a mixture thereof may be used.

In addition, the composition for stabilizing the radiochemical purity of [¹⁸F]fluoro-dopa according to the present disclosure may be supplied to a clinical syringe or container provided with a seal suitable for single or multiple perforation with a hypodermic needle while maintaining sterilizing maintainability, for example, to a single vial or a multi-use vial. The multi-use vial refers to a glass container for injection that may be used for two or more patients to use a drug included in one vial, rather than using one drug included in one vial, such as a single-person vial, for one patient and discarding the rest.

The composition for stabilizing the radiochemical purity of [¹⁸F]fluoro-dopa according to the present disclosure may be used for positron emission tomography (PET) imaging, PET/CT combined with the PET and computed tomography, or PET/MRI combined with the PET and magnetic resonance imaging.

The radiochemical purity (RCP) refers to the ratio of other radionuclides to the target radionuclide. In the present disclosure, the RCP is expressed with the activity% of [¹⁸F]fluoro-dopa relative to the total radioactivity present in a sample.

In addition, the present disclosure provides a method for preparing a composition for stabilizing the radiochemical purity of [18F]fluoro-dopa by inhibiting the formation of impurities during a predetermined time period, comprising: mixing [18F]fluoro-dopa; ethanol in a concentration of 5% to 30%(v/v) relative to the total composition; and a buffer having a pKa value of 6 to 8.1 at 25° C. The same or similar description with respect to the composition is omitted herein.

Hereinafter, the present disclosure will be described in more detail with reference to the following examples. It is to be understood, however, that the following examples are for illustrative purposes only, and the scope of the present disclosure is not limited to the following examples. In addition, it will be appreciated by a person having ordinary skill in the pertinent technical field that various modifications and variations may be made without departing from the scope and spirit of the present disclosure. It is apparent that this also falls within the scope of the present disclosure.

### EXPERIMENTAL EXAMPLE 1

### Effect of Hydrogen Ion Concentration on the Radiochemical Purity of [¹⁸F]fluoro-dopa

[¹⁸F]fluoro-dopa (radiochemical purity 100%) was prepared according to known procedures (see Appl. Radiat. Isot. 672009 1650-1653), the initial hydrogen ion concentration of the prepared [¹⁸F]fluoro-dopa (radiochemical purity 100%) is 4. This was formulated into physiological saline, and hydrogen ion concentration was adjusted to 6, 7, and 8, respectively, using sodium bicarbonate. Each time the solution was allowed to stand at room temperature (or at room temperature) for 0, 2, 4 and 6 hours, high-performance liquid chromatograph (HPLC) was used to measure the radiochemical purity. The results are listed in Table 1.

### COMPARATIVE EXAMPLE 1

### Effect of Hydrogen Ion Concentration on Radiochemical Purity of [¹⁸F]FDG

[¹⁸F]FDG (radiochemical purity 100%) was prepared according to known procedure (Ind. J. Appl. Radiatl Isot 35 1984 985-986) was formulated with physiological saline and hydrogen ion concentration was adjusted to 6, 7, and 8, respectively, using sodium bicarbonate, and then was allowed to stand at room temperature for 0, 2, 4, and 6 hours. The radiochemical purity was measured using thin film chromatography (TLC), and the results are listed in Table 1.

### COMPARATIVE EXAMPLE 2

### Effect of Hydrogen Ion Concentration on Radiochemical Purity of [¹⁸F]FLT

[¹⁸F]FLT (radiochemical purity 100%) was prepared according to known procedures (Eur. J. Nucl. Med. Mol. Imaging. 2007,34, 1406-1409) and formulated with physiological saline. Hydrogen ion concentration was adjusted to 6, 7, and 8, respectively, by using sodium bicarbonate at room temperature, and then was allowed to stand at room temperature (or at room temperature) for 0, 2, 4, and 6 hours. The radiochemical purity was measured using high-performance liquid chromatograph (HPLC), and the results are listed in Table 1. The units of the numerical values in Table 1 below are defined as %, and exhibit the changes of hydrogen ion concentration and the radiochemical purity over time based on the radiochemical purity 100% of each of the initially prepared [¹⁸F]fluoro-dopa, [¹⁸F]FDG and [¹⁸F]FLT.

TABLE 1

| | [¹⁸F]fluoro-dopa | | | [¹⁸F]FDG | | | [¹⁸F]FLT | | |
|---|---|---|---|---|---|---|---|---|---|
| | pH 6 | pH 7 | pH 8 | pH 6 | pH 7 | pH 8 | pH 6 | pH 7 | pH 8 |
| 0 hour | 100 | 100 | 99.73 | 99.07 | 99.23 | 99.78 | 100 | 100 | 99.15 |
| 2 hours | 96.66 | 92.02 | 91.14 | 98.04 | 100 | 99.01 | 100 | 100 | 100 |
| 4 hours | 92.6 | 78.67 | 76.58 | 98.12 | 99.46 | 99.89 | 99.78 | 100 | 99.29 |
| 6 hours | 87.95 | 63.81 | 51.78 | 99.12 | 98.12 | 100 | 100 | 100 | 100 |

As listed in Table 1, even when [¹⁸F]FDG of Comparative Example 1 and [¹⁸F]FLT of Comparative Example 2 were prepared with physiological saline after synthesis, they exhibited 90% or more of the radiochemical purity over time at a neutral pH (pH 6 to 8), and thus can be used as a radiopharmaceutical. Meanwhile, the radiochemical purity was rapidly decreased over time at a neutral pH (pH 6 to 8) and the [¹⁸F]fluoro-dopa of Experimental Example 1 exhibited radiochemical purity that could not be used as a radiopharmaceutical after 2 hours. This was attributed to the characteristic of [¹⁸F]fluoro-dopa, indicating that non-enzymatic oxidation occurred at room temperature and neutral pH. Thus, it could be confirmed that it is not useful as a radiopharmaceutical after 2 hours.

For PET diagnosis, [¹⁸F]fluoro-dopa should be maintained at neutral pH, not acidic, for patient administration and radiochemical purity should be maintained at room temperature for a longer period of time for storage and distribution convenience.

### EXPERIMENTAL EXAMPLE 2

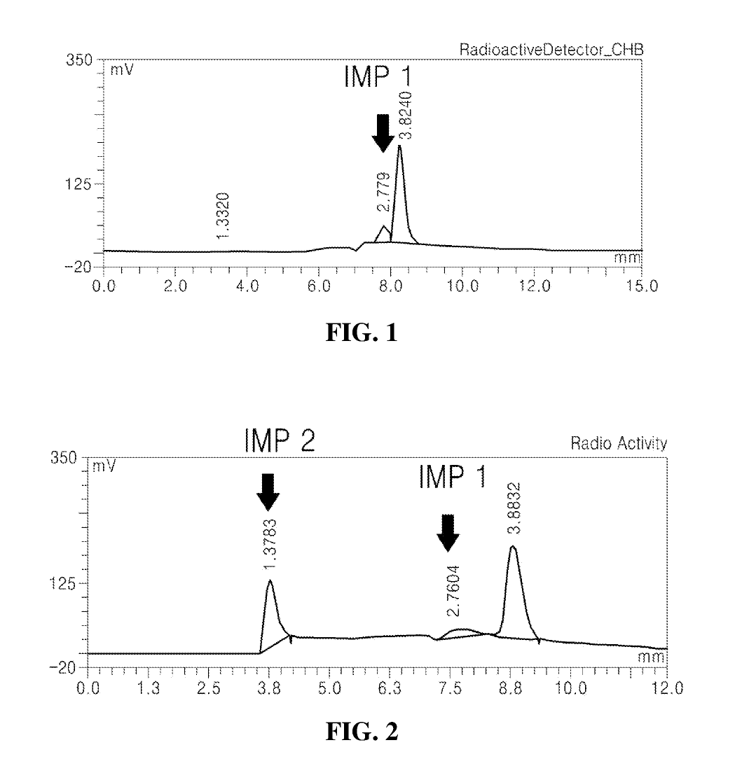

### Causes Affecting the Radiochemical Purity of [¹⁸F]fluoro-dopa

[¹⁸F]fluoro-dopa (radiochemical purity 100%) was prepared according to known procedures (see: Appl. Radiat. Isot. 67 2009 1650-1653), and the hydrogen ion concentration was adjusted to 7 (pH 7) using sodium bicarbonate after preparation with physiological saline. The determination of whether the radiochemical purity was generated was confirmed by measurement using HPLC (high-performance liquid chromatography) after 2 and 6 hours at room temperature. The results are illustrated in FIGS. 1 and 2.

FIG. 1 is an HPLC chromatogram of [¹⁸F]fluoro-dopa after 2 hours of neutralization and FIG. 2 is an HPLC chromatogram of [¹⁸F]fluoro-dopa after 6 hours of neutralization.

As illustrated in FIG. 1, it was confirmed that when 2 hours have elapsed after neutralization to pH 7 at room temperature, the first impurity (IMP 1) was generated. As illustrated in FIG. 2, it was confirmed that when 6 hours have elapsed after neutralization to pH 7 at room temperature, the first impurity (IMP 1) and the second purity (IMP 2) were generated. As listed in Table 1, the radiochemical purity of [¹⁸F]fluoro-dopa was 92.02% when 2 hours have elapsed after neutralization to pH 7 at room temperature and was significantly lowered to 63.81% after 6 hours, suggesting that as time elapses after neutralization, impurity is generated due to oxidation of [¹⁸F]fluoro-dopa, resulting in reduced radiochemical purity.

### EXPERIMENTAL EXAMPLE 3

### Effect of Ethanol on the Radiochemical Purity of [¹⁸F]fluoro-dopa

The following experiment was conducted to confirm the effect of ethanol on the radiochemical purity of [¹⁸F]fluoro-dopa in the composition of the present disclosure.

[¹⁸F]fluoro-dopa (radiochemical purity 100%) under the condition of hydrogen ion concentration 4 was prepared according to known procedures (see: Appl. Radiat. Isot. 67 2009 1650-1653) and ethanol (100% purity) were purchased from Merck. Thereafter, the [¹⁸F]fluoro-dopa was added to the prepared vial, and ethanol of each of 1.0%, 5.0%, 10.0%, 20.0%, and 30.0% (v/v) relative to the total composition was added to the vial and HPLC (high-performance liquid chromatograph) was performed at 0 hour, 2 hours, 4 hours, and 6 hours after neutralization with hydrogen ion concentrations of 6, 7, and 8 using sodium bicarbonate, respectively, to measure the radiochemical purity. By measuring the radiochemical purity, the change of radiochemical purity of [¹⁸F]fluoro-dopa with ethanol content after pH neutralization was examined.

### EXPERIMENTAL EXAMPLE 3-1

### In the Case of Neutralization with a Hydrogen Ion Concentration of 6

When neutralizing the hydrogen ion concentration to 6, the addition of 1.0%, 5.0%, 10.0%, 20.0% and 30.0% (v/v) ethanol relative to the total composition influences the radiochemical purity of [¹⁸F]fluoro-dopa as follows. As a control group, no ethanol was added (0%), and the results are listed in Table 2.

The units of numerical values in Table 2 below are percentages and exhibit the change in the amount of ethanol added and the radiochemical purity over time based on the radiochemical purity of 100% of the initially prepared [¹⁸F]fluoro-dopa.

TABLE 2

| | Ethanol | Ethanol | Ethanol | Ethanol | Ethanol | Ethanol |
|---|---|---|---|---|---|---|
| | 0% | 1% | 5% | 10% | 20% | 30% |
| 0 Hour | 100 | 100 | 100 | 100 | 100 | 100 |
| 2 hours | 96.66 | 99.29 | 100 | 100 | 100 | 100 |
| 4 hours | 92.6 | 98.61 | 100 | 100 | 100 | 100 |
| 6 hours | 87.95 | 97.82 | 100 | 100 | 100 | 100 |

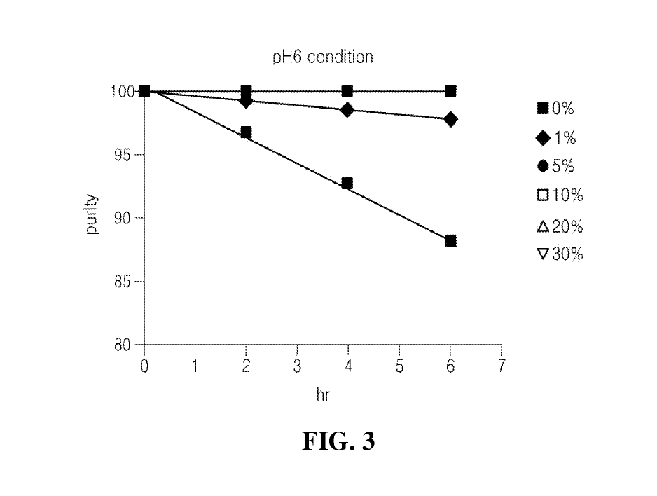

As listed in Table 2, in the case of no addition of ethanol (0%) after neutralization to pH 6, the radiochemical purity started to decrease to 96.66% from 2 hours, 92.6% after 4 hours, and 87.95% after 6 hours. After 6 hours or more, the lowest radiochemical purity, 90%, which can be used for radiopharmaceuticals, was obtained. On the other hand, when ethanol was added, it exhibited a radiochemical purity of 90% or more when neutralized at pH 6 and then 6 hours at room temperature. Especially when ethanol was added at 5% or more, it was confirmed to maintain radiochemical purity of 100% at pH6 of up to 6 hours. In addition, FIG. 3 is a graph illustrating the results of Table 2, and it is confirmed that the radiochemical purity decreases with linearity in the amount of 0 to 1% ethanol by the hour.

As a result, it can be seen that ethanol inhibits the generation of impurities due to the oxidation of [¹⁸F]fluoro-dopa over time and maintains radiochemical purity.

### EXPERIMENTAL EXAMPLE 3-2

### In the Case of Neutralization with a Hydrogen Ion Concentration of 7

When neutralizing the hydrogen ion concentration to 7, the addition of 1.0%, 5.0%, 10.0%, 20.0% and 30.0% (v/v) ethanol relative to the total composition influences the radiochemical purity of [¹⁸F]fluoro-dopa as follows. As a control group, no ethanol was added (0%), and the results are listed in Table 3.

The units of numerical values in Table 3 below are percentages and exhibit the change in the amount of ethanol added and the radiochemical purity over time based on the radiochemical purity of 100% of the initially prepared [¹⁸F]fluoro-dopa.

TABLE 3

| | Ethanol | Ethanol | Ethanol | Ethanol | Ethanol | Ethanol |
|---|---|---|---|---|---|---|
| | 0% | 1% | 5% | 10% | 20% | 30% |
| 0 Hour | 100 | 100 | 100 | 100 | 100 | 100 |
| 2 hours | 92.02 | 95.97 | 97.36 | 98.6 | 96.38 | 100 |
| 4 hours | 78.67 | 87.6 | 95.84 | 97.24 | 97.72 | 100 |
| 6 hours | 63.81 | 75.64 | 84.83 | 96.42 | 96.77 | 100 |

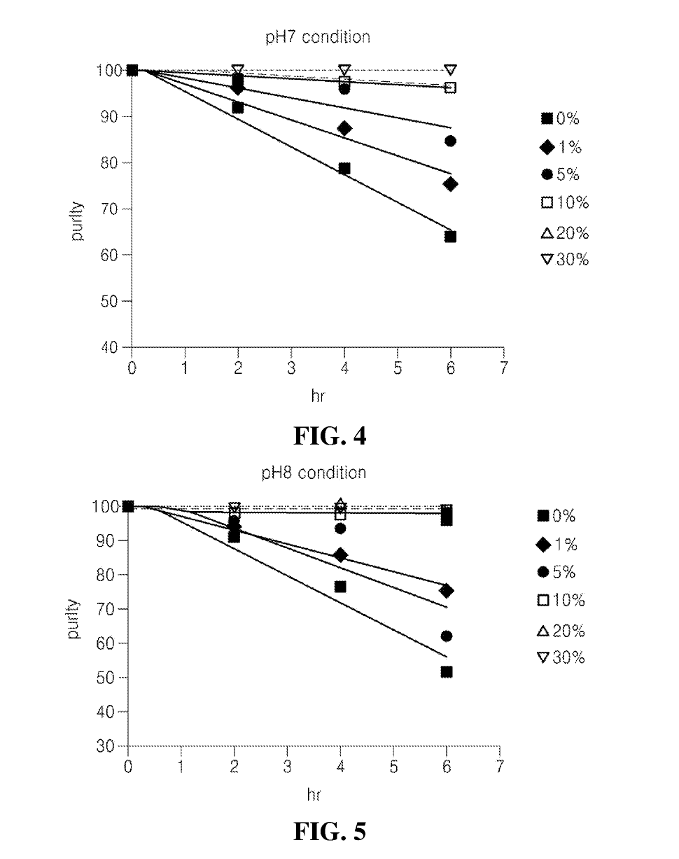

As listed in Table 3, in the case of no addition of ethanol (0%) after neutralization to pH 7, the radiochemical purity started to decrease to 95.97% from 2 hours, 87.6% after 4 hours, and 75.64% after 6 hours. After 4 hours or more, the lowest radiochemical purity, 90%, which can be used for radiopharmaceuticals, was obtained. On the other hand, when ethanol was added, the radiochemical purity was maintained at a room temperature after 6 hours of neutralization to pH 7. When 5% ethanol was added, the radiochemical purity or 90% or more was maintained until after the elapse of 4 hours. When 10% ethanol was added, it exhibited a radiochemical purity of 90% or more even after 6 hours. In addition, FIG. 4 is a graph illustrating the results of Table 3, and it is confirmed that the decrease in the radiochemical purity according to an ethanol content exhibited a linear pattern, and thus an ethanol content influences the radiochemical purity.

In addition, as exhibited in Experimental Example 2, in the case of no ethanol addition, impurity was generated after 2 hours and 6 hours at room temperature after being neutralized to pH 7, resulting in lowered radiochemical purity. In the case of ethanol addition, the radiochemical purity was maintained at 95% or more, and it can be seen that ethanol inhibits the generation of impurities due to the oxidation of [¹⁸F]fluoro-dopa over time to maintain radiochemical purity.

### EXPERIMENTAL EXAMPLE 3-3

### In the Case of Neutralization with a Hydrogen Ion Concentration of 8

When neutralizing the hydrogen ion concentration to 8, the addition of 1.0%, 5.0%, 10.0%, 20.0% and 30.0% (v/v) ethanol relative to the total composition influences the radiochemical purity of [¹⁸F]fluoro-dopa as follows. As a control group, no ethanol was added (0%), and the results are listed in Table 4.

The units of numerical values in Table 4 below are percentages and exhibit the change in the amount of ethanol added and the radiochemical purity over time based on the radiochemical purity of 100% of the initially prepared [¹⁸F]fluoro-dopa.

TABLE 4

| | Ethanol | Ethanol | Ethanol | Ethanol | Ethanol | Ethanol |
|---|---|---|---|---|---|---|
| | 0% | 1% | 5% | 10% | 20% | 30% |
| 0 Hour | 99.73 | 100 | 100 | 100 | 100 | 100 |
| 2 hours | 91.14 | 94.48 | 96.26 | 97.07 | 98.31 | 100 |
| 4 hours | 76.58 | 85.9 | 93.64 | 97.54 | 100 | 98.96 |
| 6 hours | 51.87 | 75.76 | 62.19 | 96.47 | 97.42 | 97.84 |

As listed in Table 4, in the case of no addition of ethanol (0%) after neutralization to pH 8, the radiochemical purity started to decrease to 91.14% from 2 hours, 76.58% after 4 hours, and 51.87% after 6 hours. After 4 hours or more, the lowest radiochemical purity, 90%, which can be used for radiopharmaceuticals, was obtained. On the other hand, when ethanol was added, the radiochemical purity was maintained at a room temperature after 6 hours of neutralization to pH 8. When 5% ethanol was added, the radiochemical purity or 90% or more was maintained until after the elapse of 4 hours. When 10% ethanol was added, it exhibited a radiochemical purity of 90% or more even after 6 hours. In addition, FIG. 5 is a graph illustrating the results of Table 4, and it is confirmed that the decrease in the radiochemical purity according to an ethanol content exhibited a linear pattern, and thus an ethanol content influences the radiochemical purity.

### EXPERIMENTAL EXAMPLE 4

### Influence of Storage Temperature on Radiochemical Purity of [¹⁸F]fluoro-dopa

### EXPERIMENTAL EXAMPLE 4-1

### DEFLUORINATION phenomenon

[¹⁸F]fluoro-dopa prepared by adding 5% ethanol under the same conditions as in Experimental Example 3-2 was neutralized to a hydrogen ion concentration of 7 using sodium bicarbonate, and then PET images were photographed using the same after 0 and 15 minutes at room temperature.

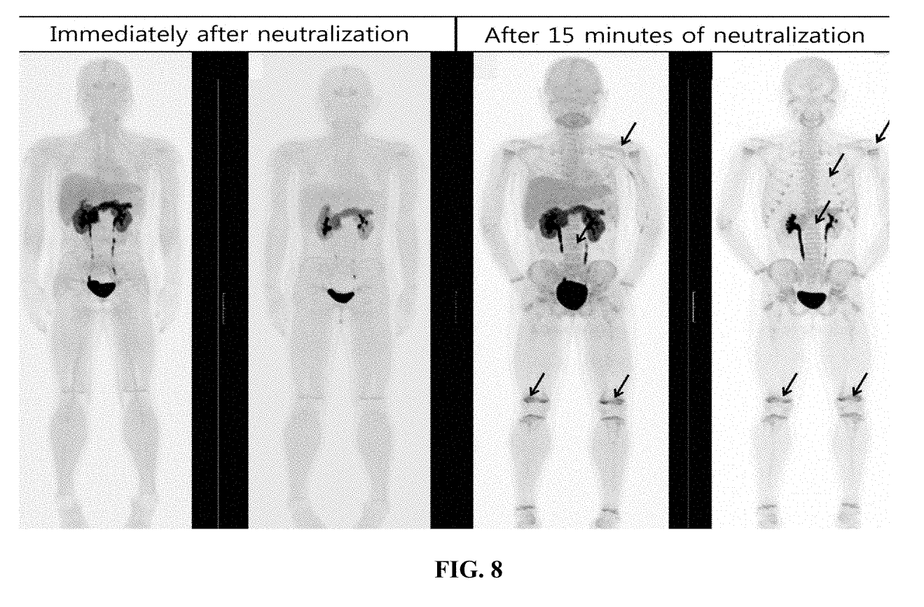

This result is illustrated in FIG. 8. The left side of FIG. 8 exhibits the results of PET imaging using [¹⁸F]fluoro-dopa preparations immediately after neutralization at room temperature, and the right side exhibits the result of PET imaging using [¹⁸F]fluoro-dopa preparations after 15 munities at room temperature after the neutralization. Upon reviewing them, the left photo could confirm an image using a normal [¹⁸F]fluoro-dopa, and in case of a right photo, the bones which are supposed to be invisible (see red arrows) are photographed in an image. In this regard, the present inventors confirmed that another impurity which was not analyzed by HPLC was present, and it was confirmed that [¹⁸F]fluoride was formed by DEFLUORINATION phenomenon when stored at room temperature. Accordingly, in the case of the photograph on the right side of FIG. 8, the bones which are supposed to be invisible due to the [¹⁸F]fluoride ingested in the bone are photographed on the PET image. This is a problem in that it is a bad image and should be retested. Thus, the [¹⁸F]fluoride (defluorine) generated by the DEFLUORINATION phenomenon is also a kind of impurity, and its production should also be inhibited.

### EXPERIMENTAL EXAMPLE 4-2

### Effect of Storage Temperature on Radiochemical Purity [¹⁸F]fluoro-dopa

In order to confirm the effect of storage temperature at room temperature on the above DEFLUORINATION phenomenon, under the same condition as the Experimental Example 3-2, 0% (no addition), 10.0% and 20.0% (v/v) ethanol relative to the total composition were added. The prepared [¹⁸F]fluoro-dopa was neutralized to a hydrogen ion concentration of 7 using sodium bicarbonate, and then the radiochemical purity exhibited after 0, 10, 20, 30, 60, 90, 120, 180, 240, 300 and 360 minutes at room temperature was measured by thin film chromatography (TLC). The reason why the TLC was used was to confirm the third impurity which was not analyzed by HPLC as in the Experimental Example 4-1.

At this time, when the ethanol was not added, it was further neutralized and stored in a refrigerator (4° C. and 2 to 8° C.) and in a freezer (−20° C.), and the radiochemical purity exhibited after 0, 10, 20, 30, 60, 90, 120, 180, 240, 300 and 360 minutes was further measured. The results are listed in Table 5.

TABLE 5

| | 0% Ethanol (no addition) | | | Ethanol 10% | Ethanol 20% |
|---|---|---|---|---|---|
| Time | Neutralization - | Neutralization - | Neutralization - | Neutralization - | Neutralization - |
| (minutes) | Room Temperature | Refrigeration | Freezing | Room temperature | Room temperature |
| 0 | 97.41 | 98.55 | 98.23 | 98.02 | 98.42 |
| 10 | 89.77 | 98.22 | 98.25 | 97.62 | 96.05 |
| 20 | 83.20 | 97.88 | 98.10 | 96.77 | 96.02 |
| 30 | 52.20 | 96.06 | 97.20 | 89.57 | 83.84 |
| 60 | 50.93 | 94.76 | 96.71 | 81.07 | 81.04 |
| 90 | 45.64 | 94.55 | 96.20 | 70.09 | 75.66 |
| 120 | 37.44 | 91.01 | 94.75 | 67.45 | 77.21 |
| 180 | 35.07 | 84.58 | 91.74 | 59.57 | 68.25 |
| 240 | 23.95 | 81.24 | 85.24 | 46.70 | 63.12 |
| 300 | 22.23 | 79.00 | 80.20 | 40.59 | 58.24 |
| 360 | 16.52 | 73.66 | 77.53 | 34.4 | 55.56 |

As listed in Table 5, the radiochemical purity was measured by TLC. As a result, in case of no addition of ethanol and storage at room temperature after neutralization, it was confirmed that the radiochemical purity was 89.77% after 10 minutes, and it rapidly decreased to 16.52% after 6 hours. On the other hand, even in case of no addition of ethanol, the radiochemical purity was 90% or less after 3 hours of neutralization when kept refrigerated, and the radiochemical purity was 90% or less after 4 hours of neutralization when kept frozen. In case of [¹⁸F]fluoro-dopa, the storage temperature affects the radiochemical purity. As a result, it was confirmed that the storage temperature affects the formation of the third impurity ([¹⁸F] fluoride).

On the other hand, in case of addition of ethanol, when 10% ethanol and 20% ethanol were added after storage at room temperature after neutralization, the radiochemical purity was 90% or less after 30 minutes of neutralization. Thus, it was confirmed that the time which indicates the radiochemical purity of 90% or less than the case of addition of no ethanol can be delayed, but the effect was not good compared with the case of refrigerated and frozen storage.

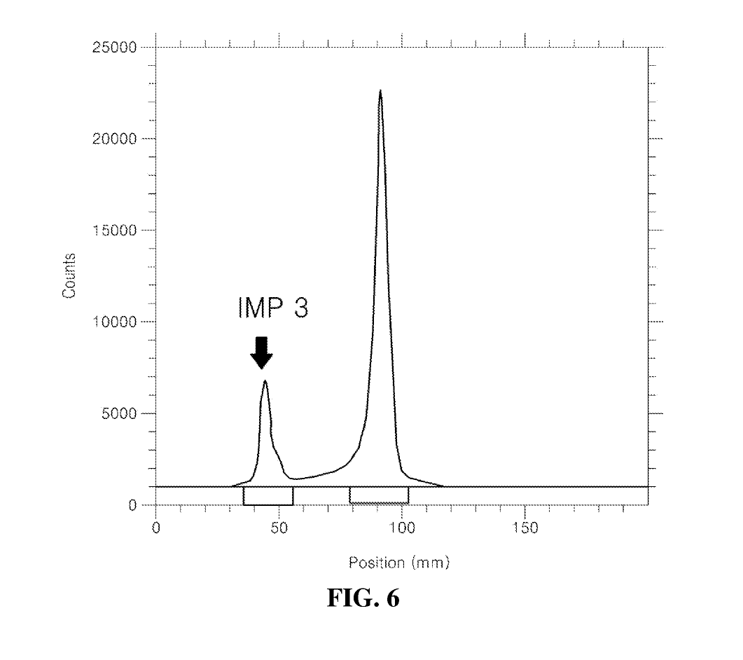

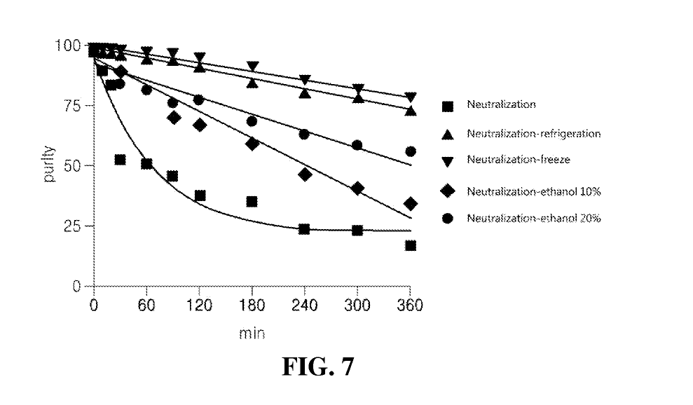

FIG. 6 is a TLC chromatogram of [¹⁸F]fluoro-dopa prepared by adding 10% of ethanol with 1 hour after neutralization. As illustrated in FIG. 6, the formation of the third impurity ([¹⁸F]fluoride, IMP3) was confirmed by TLC chromatogram. FIG. 7 is a graph illustrating the results of Table 5, and it can be seen that there is a large difference in decrease of radiochemical purity in case of refrigerated and frozen storage and storage at room temperature.

The results of Experimental Example 4-2 exhibit that the third impurity ([¹⁸F]fluoride) produced by the DEFLUORINATION phenomenon is not measured in HPLC but is regarded as a result detected in TLC. The formation of the third impurity is determined to be influenced by the storage temperature even if ethanol is added.

### EXPERIMENTAL EXAMPLE 5

### Influence on the Radiochemical Purity of [¹⁸F] fluoro-dopa in Buffer

In order to confirm the radiochemical purity which can be used as a radiopharmaceutical even at room temperature by inhibiting the formation of third impurities generated when stored at room temperature as in Experimental Example 4, an experiment was conducted to confirm whether a buffer affects the radiochemical purity of [¹⁸F]fluoro-dopa.

As in Experimental Example 3, [¹⁸F]fluoro-dopa (radiochemical purity 100%) was prepared according to known procedures under the condition of a hydrogen ion concentration 4 and added to the vial. 5.0%(v/v) ethanol relative to the total composition and a buffer having a pKa value of 6 to 8.1 at 25° C., neutralized to pH 7 with sodium bicarbonate. Then, the radiochemical purity was measured using HPLC (high purity liquid chromatography) and TLC (thin film chromatography) after 0, 10, 20, 30, 60, 90, 120, 180, 240, 300 and 360 minutes to confirm the formation of the third impurity. The results are listed in Table 6 below. The units of the numerical values in Table 6 below are defined as %, and exhibit the changes of the radiochemical purity according to the type of a buffer added with ethanol, based on the radiochemical purity 100% of the initially prepared [¹⁸F]fluoro-dopa. The sum of the HPLC measurement value and the TLC measurement value is subtracted from the initial radiochemical purity of 100%. As a result, it is possible to finally determine whether the formation of the first and second impurities measured by HPLC and the third impurity measured by TLC is inhibited.

As the buffer having a pKa value of 6 to 8.1 at 25° C., PBS(PHOSPHATE BUFFERED SALINE), CITRATE BUFFER, MES(2-(N-MORPHOLINO)ETHANESULFONIC ACID), BIS-TRIS (2,2-BIS(HYDROXYMETHYL)-2,2′,2″-NITRILOTRIETHANOL) buffer, MOPSO (3-MORPHOLINO-2-HYDROXYPROPANESULFONIC ACID) buffer, HEPES(4-(20HYDROXETHYL)-1-PIPERAZINEETHANSULFONIC ACID) buffer and TRIS(TRIS(HYDROXYMETHYL)AMINOMETHANE) buffer were used and tested for each.

TABLE 6

| Time | | | | | | | |
|---|---|---|---|---|---|---|---|
| (Min- | | | | | | | |
| utes) | PBS | Citrate | MES | Bis-Tris | MOSPO | HEPES | Tris |
| 0 | 100.00 | 99.26 | 99.34 | 99.89 | 99.78 | 99.14 | 99.76 |
| 10 | 100.00 | 99.04 | 99.08 | 99.79 | 99.84 | 98.18 | 98.65 |
| 20 | 100.00 | 98.65 | 98.45 | 99.34 | 99.57 | 99.81 | 98.76 |
| 30 | 97.41 | 99.18 | 98.75 | 97.34 | 99.04 | 98.14 | 98.04 |
| 60 | 98.62 | 98.57 | 98.67 | 98.67 | 98.00 | 97.89 | 97.65 |
| 90 | 98.91 | 98.40 | 98.57 | 98.51 | 97.59 | 98.27 | 97.14 |
| 120 | 98.72 | 98.72 | 96.75 | 96.37 | 96.57 | 97.15 | 97.86 |
| 180 | 97.41 | 96.81 | 97.38 | 97.51 | 97.48 | 96.67 | 96.37 |
| 240 | 98.62 | 97.34 | 96.34 | 95.67 | 96.97 | 95.67 | 96.48 |
| 300 | 97.66 | 96.77 | 95.37 | 95.31 | 95.76 | 96.08 | 95.71 |
| 360 | 97.51 | 95.52 | 95.72 | 95.04 | 96.14 | 95.21 | 95.07 |

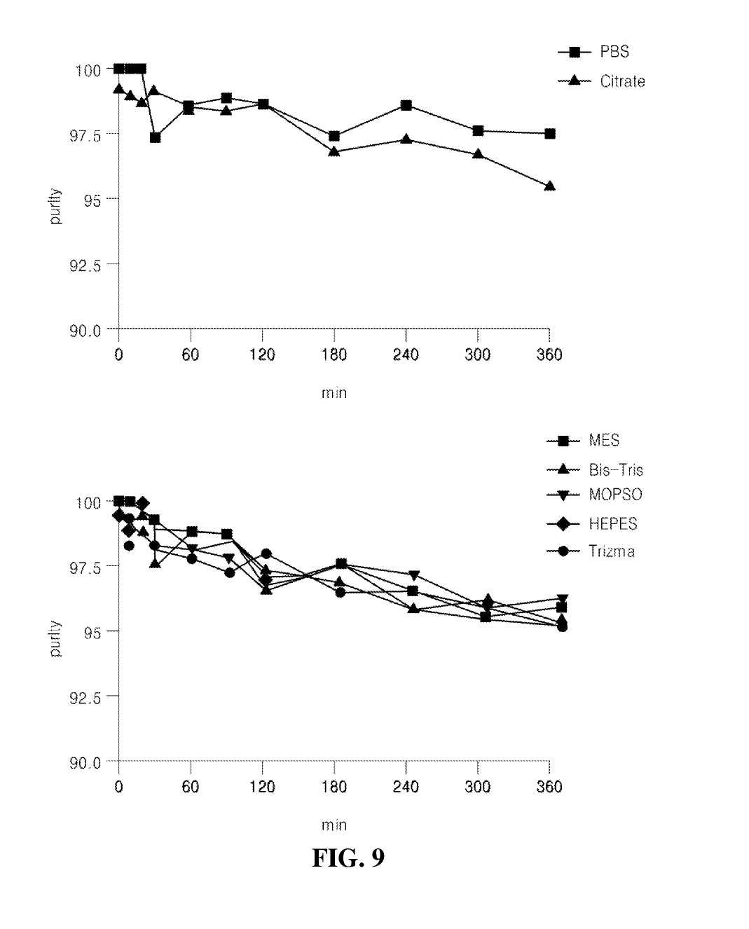

As listed in the above Table 6, it was confirmed that the buffer having a pKa value of 6 to 8.1 at 25° C. together with ethanol exhibited a radiochemical purity of 95% or more even after being stored at room temperature for 6 hours after neutralization. From this, it was confirmed that the radiochemical purity of [¹⁸F]fluoro-dopa was maintained at a high level by inhibiting the defluorinated side reaction by using the buffer and inhibiting the formation of radiochemical impurities (third impurity). Also, FIG. 9 exhibits the results of Table 6, which clearly exhibits that the radiochemical purity of [¹⁸F]fluoro-dopa is maintained at least 95% even after storage at room temperature after neutralization by using the ethanol and each buffer of the present disclosure. It was confirmed that the formation of the first to third impurities was inhibited by the ethanol and the buffer.

### INDUSTRIAL APPLICABILITY

The present disclosure may be used in a composition for stabilizing the radiochemical purity of [¹⁸F]fluoro-dopa and a method for preparing the same.

1. A composition for stabilizing a radiochemical purity of [¹⁸F]fluoro-dopa by inhibiting a formation of impurities during a predetermined time period, the composition comprising [¹⁸F]fluoro-dopa; ethanol in a concentration of 5% to 30%(v/v) relative to the total composition;
and a buffer having a pKa value of 6 to 8.1 at 25° C.

2. The composition according to claim 1, wherein the composition exhibits a radiochemical purity of at least 90% by inhibiting the formation of impurities for 2 to 6 hours after synthesis of the [¹⁸F]fluoro-dopa at a pH of 6 to 8.

3. The composition according to claim 1, wherein the composition inhibits the formation of impurities for 2 to 6 hours after synthesis of the [¹⁸F]fluoro-dopa at room temperature to exhibit a radiochemical purity of at least 90%.

4. The composition according to claim 3, wherein the buffer having a pKa value of 6.1 to 8.1 at 25° C. is at least one of those selected from the group consisting of PBS(PHOSPHATE BUFFERED SALINE), CITRATE BUFFER, MES(2-(N-MORPHOLINO)ETHANESULFONIC ACID), BIS-TRIS (2,2-BIS(HYDROXYMETHYL)-2,2′,2″-NITRILOTRIETHANOL) buffer, MOPSO (3-MORPHOLINO-2-HYDROXYPROPANESULFONIC ACID) buffer, HEPES(4-(20HYDROXETHYL)-1-PIPERAZINEETHANSFULFONIC ACID) buffer and TRIS(TRIS(HYDROXYMETHYL)AMINOMETHANE) buffer.

5. The composition according to claim 4, wherein the dopa includes levodopa (L-dopa), carbadopa, dopamine or derivatives thereof.

6. A method for preparing a composition for stabilizing a radiochemical purity of [¹⁸F]fluoro-dopa by inhibiting a formation of impurities during a predetermined time period, the method comprising: mixing [18F]fluoro-dopa; ethanol in a concentration of 5% to 30%(v/v) relative to the total composition; and a buffer having a pKa value of 6 to 8.1 at 25° C.

7. The method according to claim 6, wherein the composition exhibits a radiochemical purity of at least 90% by inhibiting the formation of impurities for 2 to 6 hours after synthesis of the [¹⁸F]fluoro-dopa at a pH of 6 to 8.

8. The method according to claim 6, wherein the composition inhibits the formation of impurities for 2 to 6 hours after synthesis of the [¹⁸F]fluoro-dopa at room temperature to exhibit a radiochemical purity of at least 90%.

9. The method according to claim 8, wherein the buffer having a pKa value of 6.1 to 8.1 at 25° C. is at least one of those selected from the group consisting of PBS(PHOSPHATE BUFFERED SALINE), CITRATE BUFFER, MES(2-(N-MORPHOLINO)ETHANESULFONIC ACID), BIS-TRIS (2,2-BIS(HYDROXYMETHYL)-2,2′,2″-NITRILOTRIETHANOL) buffer, MOPSO (3-MORPHOLINO-2-HYDROXYPROPANESULFONIC ACID) buffer, HEPES(4-(20HYDROXETHYL)-1-PIPERAZINEETHANSULFONIC ACID) buffer and TRIS(TRIS(HYDROXYMETHYL)AMINOMETHANE) buffer.

10. The method according to claim 9, wherein the dopa includes levodopa (L-dopa), carbadopa, dopamine or derivatives thereof.
